(19) United States
(12) Patent Application Publication
(10) Pub. No.: US 2023/0341422 A1
(43) Pub. Date: Oct. 26, 2023

(54) A Home Test for Measuring Glucose Control and Kidney Function in Patients

(71) Applicant: Epinex Diagnostics, Inc., Tustin, CA (US)

(72) Inventors: Bhairavi Vajaria, Tustin, CA (US); Darwin Salgado, Tustin, CA (US); Cristian Hernandez, Tustin, CA (US); Marco Robles, Tustin, CA (US); Henry Smith, Tustin, CA (US); Azra Zaidi, Tustin, CA (US)

(21) Appl. No.: 17/763,586

(22) PCT Filed: Sep. 28, 2020
(86) PCT No.: PCT/US2020/053145

§ 371 Date: Mar. 24, 2022

Related U.S. Application Data
(60) Provisional application No. 62/907,355, filed on Sep. 27, 2019.

(51) Int. Cl. G01N 33/70; G01N 33/543; G01N 33/68; B01L 3/00

(52) U.S. Cl. CPC G01N 33/70; G01N 33/5438; G01N 33/6827; B01L 3/5027

(57) ABSTRACT

The current disclosure provides a home test for simultaneous measurement of glucose control and kidney function in patients. It is a handheld device that measures the ratio of glycated albumin (GA) to albumin as well as albumin to creatinine ratio in a biological sample. The device works in conjunction with a disposable microfluidic cassette which contains an inlet for the sample to be deposited into, which might be separated into two channels or might be single channel wherein reagents are incorporated. The channels are designed to transport the sample to working electrodes coated with specific binding agents. The binding agents are antibodies or aptamers or binding peptides. The binding agents bind specifically to respective analytes in the sample and produces change in impedance. The microfluidic cassette is inserted into an instrument that automatically reads, calculates and displays GA/albumin ratio and albumin/creatinine ratio based on changes in impedance measurement.

### CROSS REFERENCE TO RELATED APPLICATIONS

The present application claims the benefit of U.S. Provisional patent application 62/907,355 filed Sep. 27, 2019, the entire contents of which is incorporated by reference herein.

### BACKGROUND

According to the 2017 National Diabetes statistics report, 30.3 million people of all ages had diabetes in 2015. The International Diabetes Federation (IDF) Diabetes Atlas (8^{th} edition) estimates that 425 million adults have diabetes and one in two remains undiagnosed, which accounts for 212 million people. Hyperglycemia can cause significant damage to some organs, which then leads to complications of diabetes like cardiac or vascular events, such as myocardial infarction (heart attack), stroke, or impaired circulation; kidney problems (nephropathy); eye problems (retinopathy); and scarring. Proper blood glucose monitoring is essential for preventing these diabetes related complications.

Traditional methods of blood glucose monitoring include measuring the concentration of blood glucose levels at the specific time of collection. Blood glucose levels fluctuate according to type of food, physical activity, and physical condition. Blood glucose monitors are routinely used by patients at home. According to a recent report, daily blood glucose monitoring is not sufficiently effective and the results convey that more than half of the study population was using the monitoring supplies incorrectly. Various factors such as application errors, extreme environmental conditions, extreme hematocrit values, or medication interferences can also influence glucose readings. Incorrect blood glucose readings may lead to treatment errors. To avoid clinical risks in response to incorrect results, appropriate patient education and diabetes management team training are mandatory.

Serum proteins, proteins that circulate in the blood such as hemoglobin (Hb) or albumin, are among the proteins affected by the glycation process. Glycated hemoglobin (HbA1c) is another test that assesses average blood glucose levels for a period of three months. However, HbA1c is not recommended in hemolytic anemia, iron deficiency anemia, hemoglobinopathies, pregnancy, or uremia. Compared to HbA1c, glycated albumin (GA) is not affected by abnormal Hb or hemolytic anemia.

Albumin is the most common protein in serum, making up about 80% of the circulating blood protein. It is recycled in the body approximately every 20-25 days and is subject to non-enzymatic glycation by excess sugar. The glycation process is a condensation reaction between carbohydrate and free amino acid at the amino terminus of proteins or the epsilon amino groups of lysine residues of proteins. Protein glycation is a marker for diabetes complications and an underlying cause of those complications. The purpose of diabetes monitoring is to help individuals with diabetes to control the glycation. Long-term control of blood glucose levels is required to lessen the damage caused by excess glucose. A glycated albumin laboratory test has the advantage of not being influenced by the concentration of other serum proteins since it is specific to the albumin glycation rates. Glycated albumin offers an advantage of monitoring average blood glucose over a short-term period of 3-4 weeks. Clinical investigations have proved that GA is a promising marker in diabetes.

Abnormal levels of GA are associated with diabetes and inflammation. Glycated albumin, along with inflammatory mediators, play an important role in insulin resistance, atherosclerosis, coronary artery disease, retinopathy, and nephropathy. Studies have shown that increased GA levels in non-diabetic patients predicts and monitors the progression of rheumatoid arthritis (RA). Glycation of albumin increases TNF-α production. TNF-α is a pro-inflammatory cytokine from adipose tissue that causes insulin resistance by enhancing adipocyte lipolysis and signaling of insulin receptor substrate. Another pro-inflammatory cytokine, IL-1β, also contributes to insulin resistance. IL-6, an inflammatory mediator also secreted by adipose tissue, causes insulin resistance by reducing the expression of glucose transporter-4 and insulin receptor substrate-1. Both GA and pro-inflammatory cytokines are involved in the pathogenesis of insulin resistance. Elevated GA levels induce insulin resistance, which in turn can lead to inflammation. Conversely, inflammation itself can lead to the development of insulin resistance. GA also plays a significant role in atherosclerosis, an inflammatory process that can eventually lead to the development of coronary artery disease. Glycated albumin is involved in the pathogenesis of diabetic retinopathy, and using agents that inhibit amadori-glycated albumin, can help to prevent this disease. Several studies indicate that GA is linked to the development of nephropathy, the thickening of the glomerular basement membrane. Glycated albumin leads to the stimulation of TGF-β production and results in increased extracellular matrix production in mesangial cells, a characteristic of nephropathy.

Considering the underlying role of GA in diabetes, retinopathy, coronary artery disease, nephropathy, and neuropathy there is an unmet need for a simplified point-of-care assay which can be used at a doctor's office or by the patient at home.

For GA testing, a presently well-known method is GlycoGap® kit(Diazyme), which is an enzymatic colorimetric assay wherein the absorbance is proportional to the concentration of glycated serum proteins or GA. An earlier lateral flow immunoassay-based point-of-care test using specific binding agents to GA and albumin, was developed for measuring GA/albumin ratio (US2014/0170766A1).

Diabetes patients are also prone to suffer from kidney disease. It is essential to monitor the kidney function along with glycemic index for monitoring diabetes. Presently, there is no home based test that helps in monitoring glucose along with kidney function.

Microalbuminuria occurs when there is an abnormal discharge of albumin from the kidneys into urine. Creatinine is a byproduct of creatinine phosphate in muscle and is excreted into urine at a constant rate. The use of the albumin-creatinine ratio (ACR) corrects for the concentration of urine, due to different hydration levels of the patient. The ACR assay serves as an early detection test for assessing kidney damage. Patients with diabetes are prone to kidney disease and hence they need to monitor ACR frequently. Increased albumin is also found in Type I diabetes, Type II diabetes, hypertension, cystic fibrosis, and diabetic nephropathy. Urine dipstick tests can be done at home and they provide a rough measure of creatinine and total protein in urine by visual assessment. However, such methods are not accurate and cannot calculate albumin/creatinine ratio. The ACR test is more accurate but is done in a laboratory and there are no home based tests by which the patient can monitor this parameter.

### SUMMARY

There is no handheld device that measures both GA/albumin and albumin/creatinine ratios simultaneously and displays both ratios. The ratio-metric analysis of GA to albumin and albumin to creatinine removes the requirement for a fixed, precise volume of sample for testing. This disclosure discloses an easy-to-use portable testing device that a person can use at home to monitor both glucose control and kidney function. It is advantageous to have a simple home based test that can be an over-the-counter product that measures both a glycemic index and kidney function.

The devices described herein measure both GA/albumin and albumin/creatinine ratios. In some embodiments, the device is handheld. In other embodiments, the devices simultaneously measure both GA/albumin and albumin/creatinine ratios. In some embodiments, both GA/albumin, and albumin/creatinine ratios are measured simultaneously by the devices described herein, which can be handheld, and the GA/albumin ratio and albumin/creatinine ratio results are displayed, for example, on a screen. The handheld device is useful to patients and health-care providers for accurate and point-of-care determination of GA/albumin and albumin/creatinine ratios. The device assists in monitoring diabetes as well as kidney function which could significantly lessen the burden of diabetes, as well as monitor the risk of kidney disease.

Disclosed herein is an easy-to-use portable testing device that a person can use at home to monitor both glucose control and kidney function. In some embodiments, the device comprises a disposable microfluidic cassette and a reusable measurable reader. The microfluidic cassette can include an inlet for the sample to be deposited into. In other embodiments, the inlet can comprise one or more channels wherein reagents are incorporated. The channels can be configured to transport the sample to one or more working electrodes where specific binding agents specific for GA, albumin, and/or creatinine are incorporated. These binding agents bind specifically to their respective analytes in the sample and produce changes in impedance. In some embodiments, the microfluidic cassette is then inserted into a reader that automatically reads, calculates and displays the GA/albumin ratio and the albumin/creatinine ratio by measuring changes in impedance. The reader can be a reusable measurable reader.

### BRIEF DESCRIPTION OF THE DRAWINGS

FIG. 1 illustrates a block diagram flow chart of the process of electrochemical measurement of GA to albumin ratio in a biosensor device.

FIG. 2 illustrates a top view of microfluidic cassette example embodiments described herein.

FIG. 3 illustrates an exemplary embodiment of a microfluidic cassette, namely Microfluidic Cassette Example Embodiment 1.

FIG. 4 illustrates an exemplary embodiment of a microfluidic cassette, namely Microfluidic Cassette Example Embodiment 4.

FIG. 5 illustrates a top view of Microfluidic Cassette Example Embodiment 1.

FIG. 6 illustrates a component breakdown from a top view for Microfluidic Cassette Example Embodiment 1.

FIG. 7 illustrates reagent integration within the channels from a top view for Microfluidic Cassette Example Embodiment 1.

FIG. 8 illustrates a disclosed microfluidic device an exploded view of a microfluidic device described herein, for example, Microfluidic Cassette Example Embodiment 1.

FIG. 9 illustrates a cross-sectional view of Microfluidic Cassette Example Embodiment 4.

FIG. 10 illustrates a cross-sectional view of the cassette reservoir of Microfluidic Cassette Example Embodiment 4.

FIG. 11 illustrates how to connect the microfluidic devices described herein with an electronic reader.

FIG. 12 illustrates an exemplary Nyquist plot.

FIG. 13 illustrates the relationship between impedance and concentration values.

FIG. 14 illustrates the logarithmic linear relationship between impedance and concentration.

FIG. 15 illustrates overlapping Nyquist curves for different concentrations of HSA standards in the microfluidic devices described herein.

FIG. 16 illustrates overlapping graphs of charge transfer resistance Rct (in Ohms) and HSA standard concentration (mg/ml) for three electrodes: (A) working electrode, (B) auxiliary or counter electrode, and (C) reference electrode in the microfluidic devices described herein.

FIG. 17 illustrates Nyquist curve overlays of different concentration curves of creatinine in the microfluidic devices described herein.

FIG. 18 illustrates graphs of overlays of electrodes A, B, and C with different concentrations of creatinine in the microfluidic devices described herein.

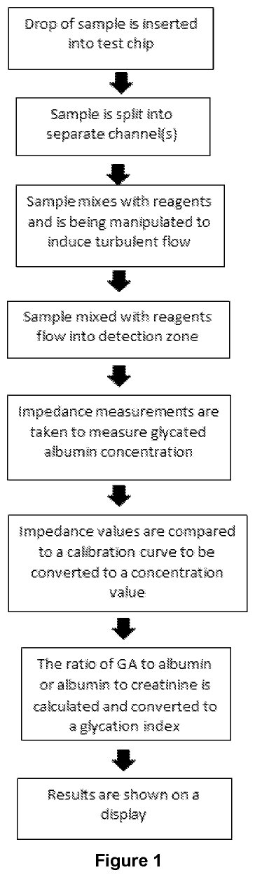

### DETAILED DESCRIPTION

Disclosed herein is a point-of-care test which produces a quantitative measurement of GA to albumin ratio and albumin to creatinine ratio in biological fluids using a handheld instrument that reads and displays the result. It uses a disposable microfluidic cassette that is inserted into the instrument while requiring only a small sample volume to perform the test. In some embodiments, the sample can be a fluid. In other embodiments, the sample can be blood or urine depending on the analytes being tested. Sample and fluid are used interchangeably throughout.

The devices described herein measure glucose control and kidney function. In some embodiments, a device for measuring glucose control and kidney function comprises a microfluidic cassette and reader, wherein the microfluidic cassette is inserted into the reader and a ratio is displayed on the screen of the reader. In other embodiments, a device for measuring glucose control and kidney function comprises a microfluidic cassette comprising an inlet including at least one channel, one or more reagents, one or more binding agents, and one or more electrode(s); a fluid sample; and a reader wherein the microfluidic cassette is inserted into the reader and displays a ratio of glycated albumin to total albumin in the fluid sample by measuring changes in impedance.

In other embodiments, the ratio is of albumin to creatinine, or glycated albumin to total albumin and albumin to creatinine, or any combination thereof.

In some embodiments, the fluid sample is deposited into the inlet. The inlet can comprise one, two, three, four, five, six, seven, eight or more channels. The sample is deposited into the inlet and then configured to pass through the number of channels created by the inlet. For example, in some embodiments the inlet divides into two separate channels, and the fluid sample flows from the inlet into the two separate channels. In other embodiments, the device comprises two inlets which create and divide into four separate channels, and the fluid sample flows from the inlet into the four separate channels.

The channels can include one or more reagents. In some embodiments, the channels are configured to measure glycated albumin, total albumin, creatinine, and/or any combination thereof. In other embodiments, a channel can have a proximal end and a distal end. The channel(s) propel the fluid sample forward until it reaches the electrodes which are located at the distal end of the channel(s). In some embodiments, the electrodes can be located at the proximal end of the channel(s) or in between the proximal and distal end of the channel. The electrodes can comprise one or more binding agents. The binding agents are specific for glycated albumin, creatinine, albumin, and/or a combination thereof. In some embodiments, a reaction between the fluid sample, one or more binding agents, and one or more reagents generate a change in impedance which is measured to display a ratio on the reader.

The microfluidic cassette comprises various example embodiments as mentioned below:

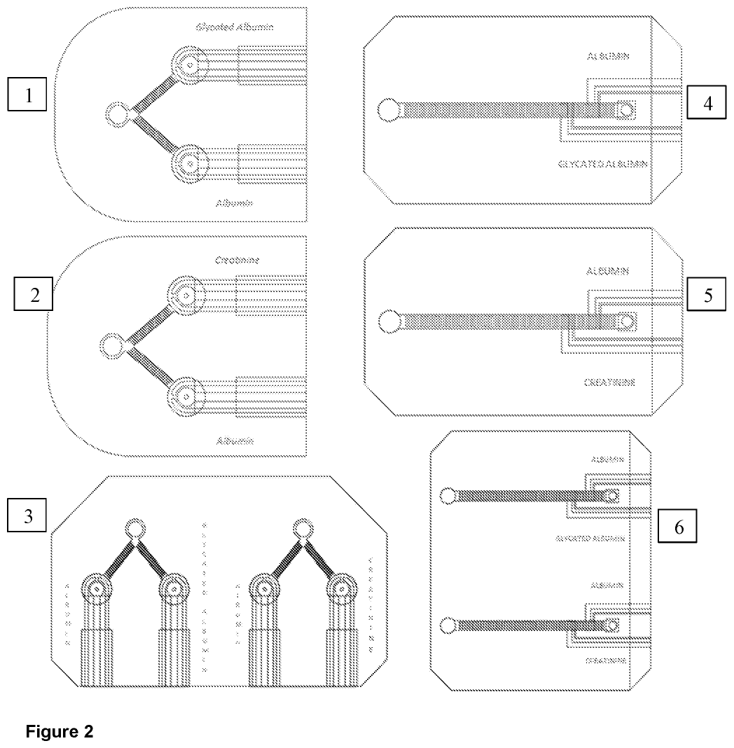

Microfluidic Cassette Example Embodiment 1: FIG. 2(1) illustrates Microfluidic Cassette Example Embodiment 1, which can measure GA/albumin. It can comprise an inlet, a membrane, at least one channel, reagent(s), binding agent(s), and electrode(s). This embodiment can include two channels, wherein one channel is configured to measure GA and the other channel is configured to measure albumin. The channels propel the sample forward until it reaches the electrode that is arranged at the end of the channel structure. Electrode(s) can include, but are not limited to, a working electrode, an auxiliary electrode, a counter electrode, a reference electrode and/or a combination thereof.

Microfluidic Cassette Example Embodiment 2: FIG. 2(2) illustrates Microfluidic Cassette Example Embodiment 2, which can measure albumin/creatinine. It can comprise an inlet, a membrane, at least one channel, reagent(s), binding agent(s), and electrode(s). This embodiment can comprise two channels, wherein one channel is configured to measure albumin and the other channel is configured to measure creatinine. The channels propel the sample forward until it reaches the electrode that is arranged at the end of the channel structure. Electrode(s) can include, but are not limited to, a working electrode, an auxiliary electrode, a counter electrode, a reference electrode and/or a combination thereof.

Microfluidic Cassette Example Embodiment 3: FIG. 2(3) illustrates Microfluidic Cassette Example Embodiment 3, which can measure GA/albumin and albumin/creatinine. It can comprise two inlets, a membrane, four channels, reagent(s), binding agent(s), and electrode(s), wherein each inlet includes two channels. In this embodiment, one inlet includes two channels for measuring GA and albumin respectively, and the other inlet includes two channels for measuring albumin and creatinine respectively. The channels propel the sample forward until it reaches the electrode that is arranged at the end of the channel structure. Electrode(s) can include, but are not limited to, a working electrode, an auxiliary electrode, a counter electrode, a reference electrode and/or a combination thereof.

Microfluidic Cassette Example Embodiment 4: FIG. 2(4) illustrates Microfluidic Cassette Example Embodiment 4, which can measure GA/albumin. It can comprise an inlet, a membrane, one channel reagent(s), binding agent(s), and electrode(s), wherein the one channel measures GA and albumin simultaneously. The channel propels the sample forward until it passes the electrode(s) that are arranged in the channel, while the waste goes to the reservoir. Electrode(s) can include, but are not limited to, a working electrode, an auxiliary electrode, a counter electrode, a reference electrode and/or a combination thereof.

Microfluidic Cassette Example Embodiment 5: FIG. 2(5) illustrates Microfluidic Cassette Example Embodiment 5, which can which measure albumin/creatinine. It can comprise an inlet, a membrane, one channel, reagent(s), binding agent(s), and electrode(s), wherein the one channel measures albumin and creatinine simultaneously. The channel propels the sample forward until it passes the electrodes that are arranged in the channel, while the waste goes to the reservoir. Electrode(s) can include, but are not limited to, a working electrode, an auxiliary electrode, a counter electrode, a reference electrode and/or a combination thereof.

Microfluidic Cassette Example Embodiment 6: FIG. 2(6) illustrates Microfluidic Cassette Example Embodiment 6, which can measure GA/albumin and albumin/creatinine. It can comprise an inlet, a membrane, two channels, reagent(s), binding agent(s), and electrode(s), wherein one channel measures GA and albumin simultaneously, and other channel measures albumin and creatinine simultaneously. The channels propel the sample forward until it passes the electrode(s) that are arranged in the channel, while the waste goes to the reservoir. Electrode(s) can include, but are not limited to, a working electrode, an auxiliary electrode, a counter electrode, a reference electrode and/or a combination thereof.

In some embodiments, the example embodiments implement passive mixing, meaning that the sample moves via capillary action with no external power source to propel the fluid forward. In other embodiments, the channel(s) can include small ridges configured to generate turbulent flow for improved mixing. The channel configuration allows the sample to be transported to the electrode(s) onto which binding agents are coated. The reaction between the sample, binding agent(s), and reagent(s) generates a change in impedance. The ratio between the impedance change for GA/albumin or albumin/creatinine is then measured, analyzed, and displayed on an electric reader's monitor.

The design embodiments of the microfluidic cassette example embodiments are discussed below:

In some embodiments, the microfluidic cassette intakes the sample, divides it into separate channels, mixes it with reagents necessary for electrochemical measurement, and transports it to working electrodes with immobilized binding agents specific for albumin, creatinine, or GA. The instrument then measures the change in impedance and displays the desired ratiometric: GA/albumin ratio, albumin/creatinine ratio, or both ratios simultaneously.

The microfluidic cassette example embodiments function similarly with differences being among the design configuration, number of channels, and electrode arrangement. The microfluidic cassette example embodiments function by transporting the sample via a channel structure. The sample mixes with the necessary reagents, and the channel leads the mixed sample to the working electrodes containing binding agents specific to GA, albumin, or creatinine. Another differences, is that microfluidic cassette example embodiments 1-3 include channels that transport the fluid to electrodes, while microfluidic cassette example embodiments 4-6 include the electrodes integrated in the channels, and include a reservoir.

In some embodiments, the electrodes are r located where the channel configuration ends. This configuration is depicted in FIG. 2, Microfluidic Cassette Example Embodiments 1-3. In other embodiments, the electrodes are vertically integrated and patterned along a channel. This configuration is depicted in FIG. 2, Microfluidic Cassette Example Embodiments 4-6.

In some embodiments, a serpentine channel is included to assist in mixing the reagents with the sample.

In other embodiments, as the sample flows along a channel, turbulent flow is induced via surface modifications of the channels to facilitate passive mixing.

In some embodiments, channels are modified with structures designed to disrupt laminar flow, such as but not limited to, ridges, herringbone structures, fins, etc.

Device Components

In some embodiments, the designs described herein implement passive mixing, meaning that the fluid mixes via capillary action with no external power source to propel the fluid forward. In other embodiments, the channel(s) include small ridges configured to generate turbulent flow for better mixing. In some embodiments, the ridges are at or near the top of the channel; in other embodiments, the ridges are implemented at the bottom of the channel. The channel configuration allows the sample to be transported to electrodes that have been coated with binding agents. The reaction occurs between the sample and binding agents coated on the electrodes, which generates a change in impedance. The ratio between impedance change for GA/albumin and albumin/creatinine in various configurations of channels is then measured, analyzed, and displayed on an electric reader's monitor.

Inlet

In some embodiments, the sample is deposited via a blood droplet directly onto an inlet located on C1, as seen in FIG. 3(7), to initiate the test. The sample can be deposited by applying it directly from the finger upon drawing blood using a lancet for a measurement of GA/albumin. In other embodiments, the sample can be deposited on the side of the microfluidic cassette by placing a fingertip on the edge. In yet other embodiments, the sample can be dispensed into the inlet by a disposable pipette, which can optionally be provided along with the microfluidic cassette.

Red blood cells (RBC) may need to be filtered out and removed as they can interfere with GA/albumin determination. In some embodiments, after the sample is deposited in sample inlet, it comes in contact with a porous membrane, which functions to filter out RBCs and allows only serum/plasma to pass through. In some embodiments, the membranes are asymmetric, meaning that the pore size decreases progressively from the top surface to its bottom. The membranes' appropriate pore size allows GA and albumin to pass through onto the microchannel structure.

In another embodiment, the sample is urine and the urine is applied into inlet by means of droplet.

FIG. 3 displays Biosensor Microfluidic Device Version 1 (V1), were the design configuration includes Microfluidic Cassette Example Embodiments 1, 2, and 3 from FIG. 2. The reference numbers pertain to the following components:
7: Inlet—sample is introduced via the inlet.
8: Vents—design allows air to flow through, but prevents the sample from exiting.
9: Channel—one channel that further splits into more channels based on the configuration/example embodiment.
10: Filtration Membrane—Filters RBCs from the sample to improve binding.
11: Turbulent Ridges—The ridge configuration generates turbulent flow to increase mixing.
12: Reagents—Can be dried, immobilized, strips, or liquid.
13: Electrode—The electrodes are screen printed and coated with the binding agents.
14: Bottom Cap—Has cutouts that expose electrodes to connect it with the reader.

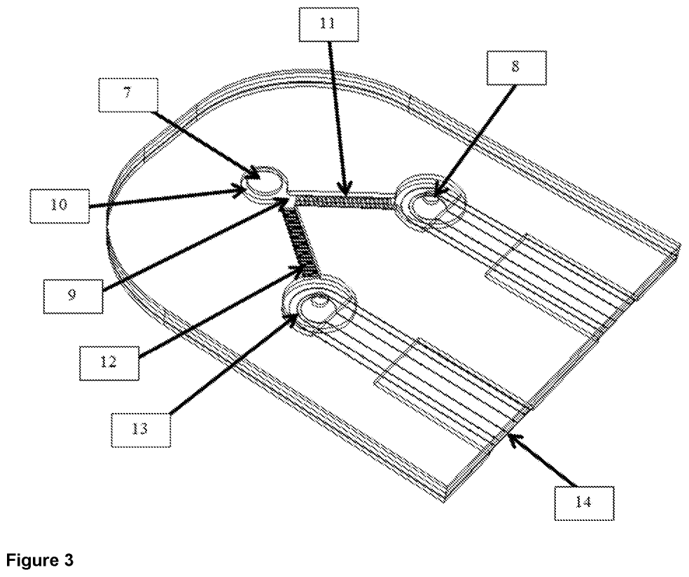

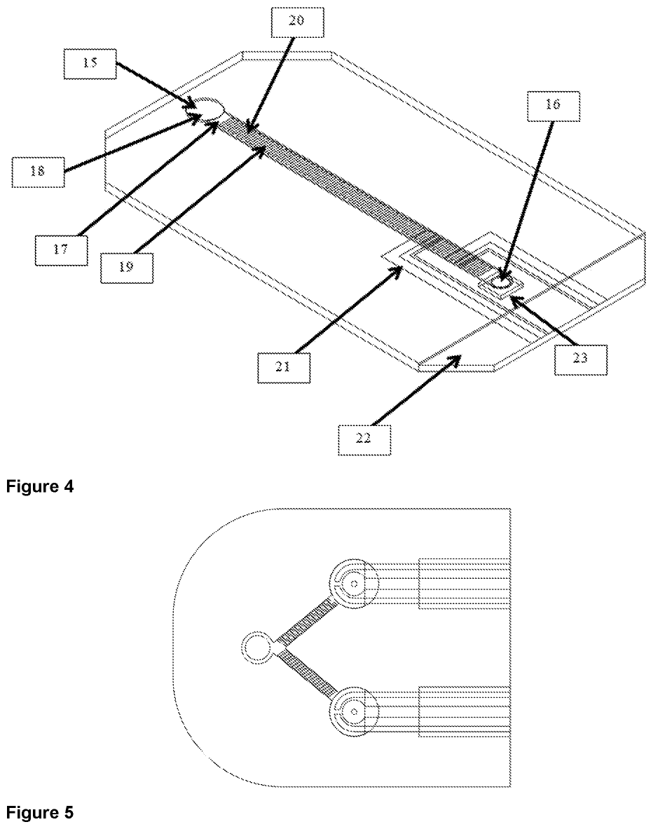

FIG. 4 displays Biosensor Microfluidic Device Version 4, were the design configuration includes Microfluidic Cassette Example Embodiments 4, 5, and 6 from FIG. 2.
15: Inlet—sample is introduced via the inlet.
16: Vents—design allows air to flow through, but prevents the sample from exiting.
17: Channel—The design configuration is one channel only.
18: Filtration Membrane—The membrane filters the sample to remove RBCs.
19: Turbulent Wells—The well configuration generates turbulent flow to increase mixing.
20: Reagents—Can be dried, immobilized, strips, or liquid depending on testing.
21: Electrode—The electrodes are screen printed and coated with the binding agents.
22: Bottom Cap—Has cutouts that expose electrodes to connect it with the reader.
23: Waste Reservoir—A reservoir that stores excess fluid waste.

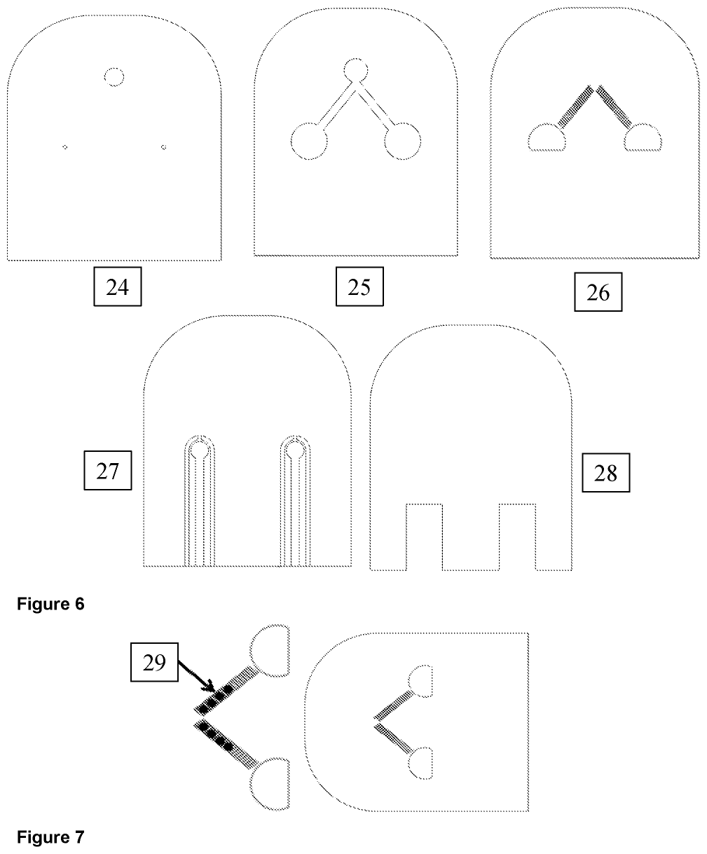

FIG. 5 depicts a top view of V1 assembled, and FIG. 6 depicts components 24, 25, 26, 27, and 28 are the Biosensor V1 Component Breakdown, V1 microfluidic device design comprises, but is not limited to, five components, such that the component breakdown yields the design functionality of said components:
24: Component 1 (C1) comprises one fluid inlet and two vent systems that allow air, but not fluid, to flow out (FIG. 6).
25: Component 2 (C2) comprises two channels which guide the fluid via capillary action, in order to reach two specific binding agent locations for GA or albumin coated on electrodes (FIG. 6).
26: Component 3 (C3) comprises ridges arranged in the channels to generate turbulent flow to improve mixing within the channels. C3 has outlets that allow the fluid to flow onto the next layer (FIG. 6).
27: Component 4 (C4) comprises screen-printed electrodes that house the binding agents and connect to the electronic reader to analyze the change in impedance, analyze the data and compare with calibration curve(s) to measure the concentration of GA/albumin or albumin/creatinine, and display the result output in the form of a GA/albumin ratio or albumin/creatinine ratio (FIG. 6).
28: Component 5 (C5) includes two cut-outs that align with the electrodes to allow for easier connection between the device and electronic reader (FIG. 6).
29: Displays the reagent incorporation in channels (FIG. 7).

FIG. 8 depicts the biosensor microfluidic V1 exploded view of the components.
30: Membrane—Exploded view showing where the membrane is located.
31: Reagents—Exploded view showing where reagents are located.
32: Ridges—Cross-sectional view of ridges within the channel that induce turbulence.
33: Electrodes—Cross-sectional view of integrated electrodes.
34: Reagent—Cross-sectional view of a section where reagents are located.
35: Waste Reservoir—A reservoir that stores excess fluid waste.

In some embodiments, the cassettes have electrodes at the distal end of the channel that come in direct contact with a handheld electronic reader that analyzes, converts, and displays the data onto a user friendly device.

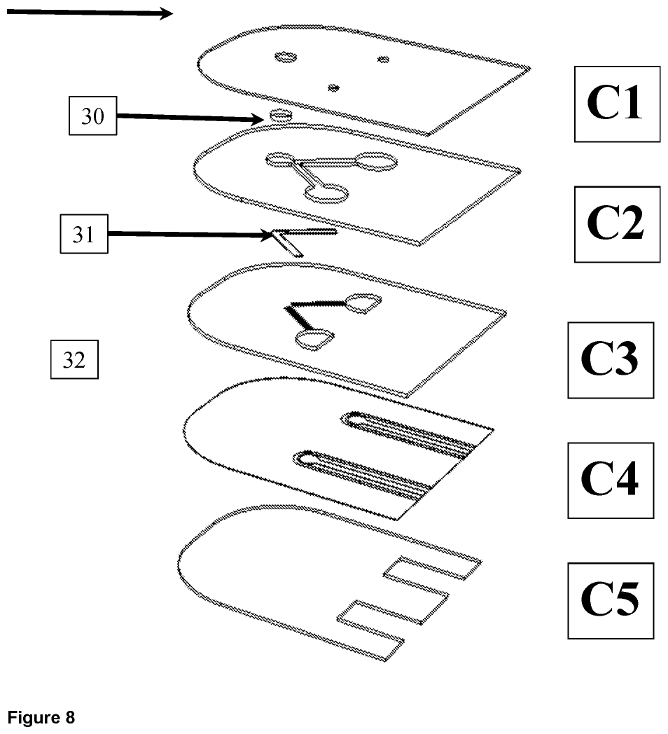

FIG. 9 shows a cross-sectional view of Microfluidic Cassette Example Embodiments 4, 5, and 6 from FIG. 2 in which the electrodes are integrated along the channel. The electrodes are incorporated within the channels as opposed to the end of the channels to increase sample reading accuracy and sensitivity (see FIG. 9(33)).

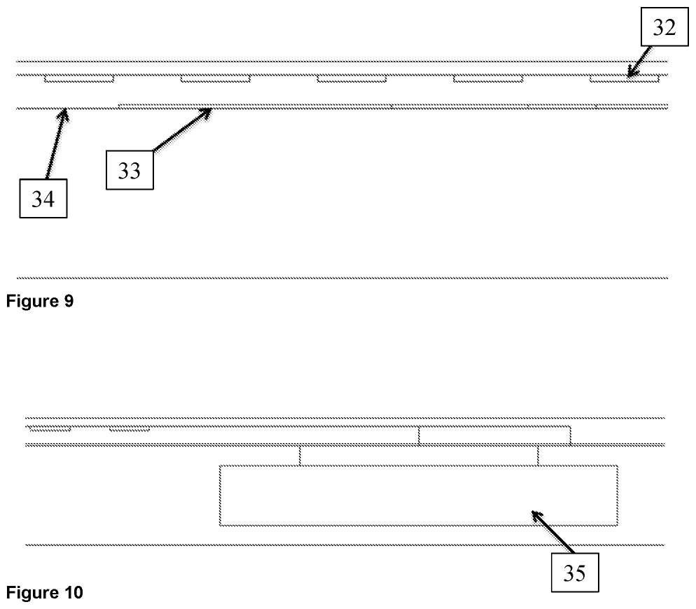

FIG. 10 shows a cross-sectional view of Microfluidic Cassette Example Embodiments 4, 5, and 6 from FIG. 2 that have a waste reservoir. The channel leads to the reservoir to dispense excess fluid that has already passed through the electrodes (see FIG. 10(35)).

FIG. 11 depicts how to connect the microfluidic cassette with the electronic reader. FIG. 11(36) depicts the location in which the cassette is required to be inserted. FIG. 11(37) depicts the connection between the microfluidic cassette and electronic reader.
36: Cassette Insertion—The cassette is inserted at the bottom of the electronic reader.
37: Product Connection—Depiction of connection between cassette and reader.

Reagent Incorporation:

In some embodiments, blood or urine are diluted in suitable diluent before dispensing in a microfluidic cassette inlet.

In some embodiments, the inlet is separated into one, two, three, four, or more channels. Within the channels the sample comes into contact with lyophilized reagents, which include but are not limited to phosphate buffered saline (PBS) containing one or more of magnesium chloride (MgCl₂), sodium chloride (NaCl), potassium chloride (KCl), potassium nitrate (KNO₃), and ferric/ferrocyanide [Fe(CN)₆]^{-3/-4}.

The reagents are wetted by the sample and mix with the sample to create the conditions necessary to generate the electrical impedance change.

In some embodiments, the lyophilized reagent layer may be deposited on the cassette in the form of a spray.

The reagents are dried, immobilized, membrane strips, or in liquid form. The sample together with reagents is transported to a detection zone, where it comes into contact with the electrodes. The surface of the working electrode is deposited with gold nanoparticles or nanotubes. The binding agents (antibodies, aptamers, or binding peptides) are immobilized on the modified working electrode by self-assembly.

Binding Agents to Coat the Electrodes

There are essentially three different types of binding agents that can be used to coat the electrode. They can be categorized as antibodies, binding peptides, and aptamers. They all perform essentially the same function of binding to their respective ligands.

A) Antibodies

There are several different types of antibodies that can be used for incorporation onto the electrodes. Polyclonal antibodies are produced by immunization, monoclonal antibodies by using hybridoma technology, and recombinant antibodies by using genetic engineering techniques. In this disclosure the term “antibody” is used to include the whole antibody molecule and/or the binding fragment of the antibody molecule.

Polyclonal antibodies are produced by immunizing various species of animals such as rabbits, goats and horses with the antigen. The polyclonal antibodies are purified using standard laboratory techniques such as salt-fractionation, gel-filtration and affinity chromatography methods. These and other methods of developing and purifying antibodies are known to those skilled in the art and are included within the scope of this disclosure.

Monoclonal antibodies are known to those of skill in the art and are included within the scope of this disclosure. Typically, monoclonal antibodies are produced using murine hybridoma technology. In order to avoid exposing the patient to foreign proteins (e.g. murine antibodies) the monoclonal antibodies are often “humanized” by replacing certain portions of the mouse antibody protein with human material. There are also newer means of producing fully human monoclonal antibodies using phage display and genetic engineering technology. For an ex vivo application such as in the herein disclosed device, any of these types of antibodies are acceptable.

All these methods of producing antibodies are known to those of skill in the art and are within the scope of this disclosure.

B) Binding Peptide

Another type of binding agent that closely mimics the action of an antibody is a binding peptide. There are various methods for preparing synthetic or biological peptide libraries composed of up to a billion different sequences, and for identifying a particular peptide sequence that targets a particular protein. Typically a large number of different peptide sequences are allowed to react with the antigen. Once the binding peptide sequence is identified, increased quantities of that binding peptide can be produced by synthesis or using genetic engineering technology. The means of producing synthetic or biologically derived peptides are known to those of skill in the art and are within the scope of this disclosure.

C) Aptamers

Aptamers are small (i.e. 40 to 100 bases), synthetic oligonucleotides. They may be composed as a single-stranded DNA chain (ssDNA) or a single-stranded RNA chain (ssRNA). Each aptamer has a unique configuration as a result of the composition of the nucleotide bases in the chain causing the molecule to fold in a particular manner. Because of their folded structure each aptamer binds selectively to a particular ligand in a manner analogous to an antibody binding to its antigen. Aptamers are able to specifically recognize and bind to virtually any kind of target, including ions, whole cells, drugs, toxins, low-molecular-weight ligands, peptides, and proteins.

Aptamers are usually synthesized from combinatorial oligonucleotide libraries using in vitro selection methods such as the Systematic Evolution of Ligands by Exponential Enrichment (SELEX). This is a technique used for isolating functional synthetic nucleic acids by the in vitro screening of large, random libraries of oligonucleotides using an iterative process of adsorption, recovery, and amplification of the oligonucleotide sequences. The iterative process is carried out under increasingly stringent conditions to achieve an aptamer of high affinity for a particular target ligand. Once the nucleotide sequence is identified increased quantities of that aptamer can be synthesized. In order to improve stability or improved binding characteristics the aptamer may be modified in various ways such as for example being synthesized as L-nucleotides instead of D-nucleotides and/or attaching branched chains to the original oligonucleotide chain.

Since the SELEX was first introduced a variety of other methods and variations of producing aptamers have been developed. These methods are known to those of skill in the art and are within the scope of this disclosure. In this disclosure the term “aptamer” refers to ssDNA aptamers and ssRNA aptamers and all modifications to the original oligonucleotide chain.

In some embodiments, the aptamers might be bound with poly(ethylene glycol) (PEG) to increase aptamer stability.

The binding agents are conjugated to the electrode by avidin/biotin technology. Avidin is a basic tetrameric glycoprotein composed of four identical subunits, each binds to biotin with high specificity and affinity. Compared to other interactions, the avidin-biotin system provides enormous advantages such as amplification of weak signals, efficient operation, highly stability and enables the use of highly diluted primary antibodies. The most widely used analogue of avidin is streptavidin. In some embodiments, the electrode is coated with streptavidin and biotinylated binding agents (antibodies, aptamers or binding peptides) are then linked to streptavidin.

After the binding agents are immobilized, the electrodes can also be treated with 6-mercap-1-hexanol (MCH) to reduce nonspecific adsorption and reduce false positives.

Electrodes/Impedance Measurement

Electrode materials can include, but are not limited to, gold, or carbon, or graphene for the working electrode, platinum/carbon for the auxiliary (counter) electrode, and Ag/AgCl for the reference electrode.

In some embodiments, the gold electrode is impregnated with gold nanoparticles or nanotubes along with streptavidin. The biotinylated binding agent (antibodies, aptamers or binding peptides)is immobilized on the modified working electrode by self-assembly.

The measurement procedure involves sample addition to the microfluidic cassette inlet. Next the sample reaches working electrodes coated with immobilized aptamers, or, antibodies, or binding peptides specific to GA, albumin, or creatinine. Before and after incubation with sample, the impedance is recorded and the concentration is then calculated by correlating with standard curve plotted earlier using standard solutions of albumin and GA.

In the presence of GA/albumin/creatinine, the binding agent folds around the analyte and forms a 3D complex that can disrupt electron transfer between ferric/ferrocyanide and the working electrode. This change in impedance can be determined from the electrodes and measured.

In some embodiments, the concentration of analyte is measured by monitoring the change of impedance, which is a direct correlation of analyte bounded to the binding agents immobilized on the working electrode. As the number of analyte molecules bound to the binding agents increases, the electron transfer between ferric/ferrocyanide and the working electrode is disturbed. In other words the impedance increases as more analyte binds with the binding agents.

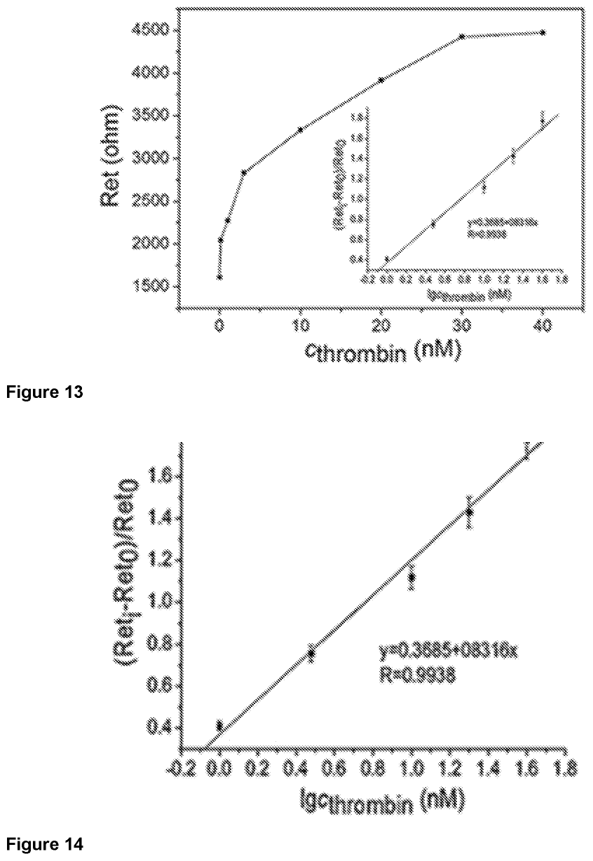

An example of the relationship between impedance and analyte concentration is shown in FIG. 13, and the logarithmic linear relationship is shown in FIG. 14. Parameters such as frequency, voltage, current, etc. can be optimized to receive the best signal possible.

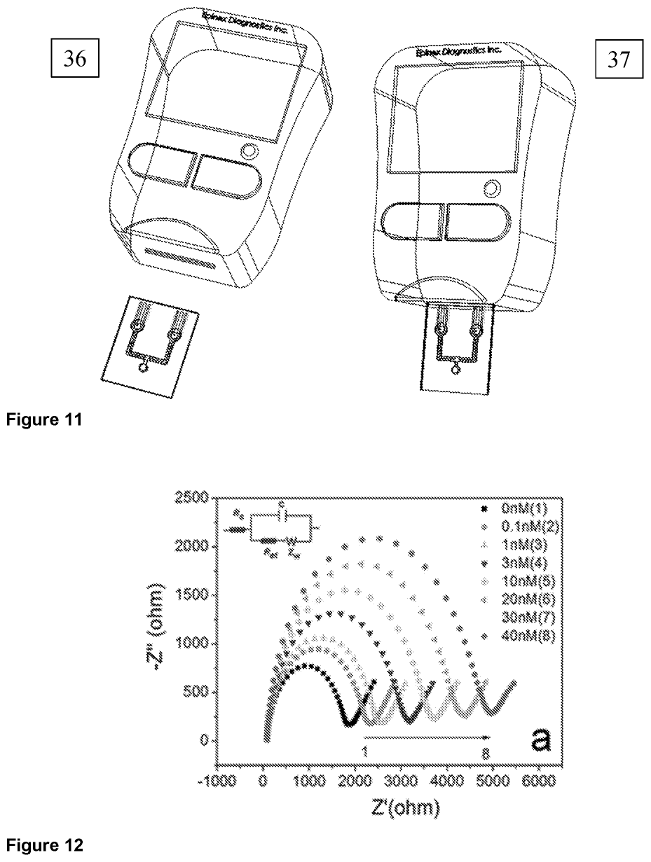

In some embodiments, electrical impedance spectroscopy can be performed to measure impedance across a broad range of frequencies. An example of a Nyquist plot generated from this technique is shown in FIG. 12. The radius of the semicircles in the plot increases with higher impedance values and hence higher concentration values. Nyquist plots can be automatically generated with data acquisition and analysis software.

In another embodiment, impedance can be measured over time with a certain frequency, voltage amplitude, current, and DC bias voltage.

In some embodiments, other electrochemical measurement techniques or voltammetry can be used to measure a change in current or impedance as the analyte binds to the binding agent. Such as but not limited to: square wave voltammetry, differential pulse voltammetry, faradaic impedance spectroscopy, cyclic voltammetry, etc.

Microfluidic Cassette Reader

The method for monitoring glycation control and kidney function described herein includes the use of a handheld and user friendly device that is able to perform multiple ratiometric tests by electrochemical means. Different test cassettes that are designed for this particular device can utilize the same electrochemical measurement technique to measure the ratio of two or more analytes in the sample.

In some embodiments, electrical impedance measurements are taken to calculate the concentration of an analyte. Electrical impedance spectroscopy (EIS) in particular, is a common technique used in the art. Those skilled in the art understand that the fundamental components needed to measure the concentration of an analyte using impedance measurements require an electrode system that has specific binding agents immobilized on the working electrode, a redox mediator present in the detection solution to facilitate electron transfer (e.g. ferric/ferrocyanide with phosphate buffer solution), and a sample that has the analyte of interest. As analytes bind to the binding agents a 3-dimensional complex is formed on the surface of the working electrode that creates resistance for electron transfer between the redox mediator and the working electrode.

In some embodiments, the same reagents and electrochemical technique would be used in each type of test cassette specific for measuring different analytes. The difference between measuring one particular analyte from another would be using a different binding agent that is specific for that analyte. Since all the test cassettes implement the same chemistry associated with measuring the concentration of analyte, the device performs that specific voltammetry, amperometry, or impedance measurement for each ratiometric test that may only vary slightly in electrical parameters applied such as voltage, current, frequency, etc.

In some embodiments, the device stores multiple calibration curves and reference data specific to each analyte measurement in a microprocessor, microcomputer, or memory chip. Furthermore a form of identification code specific to each test cassette is recognized by the device to use the appropriate calibration curve and reference data.

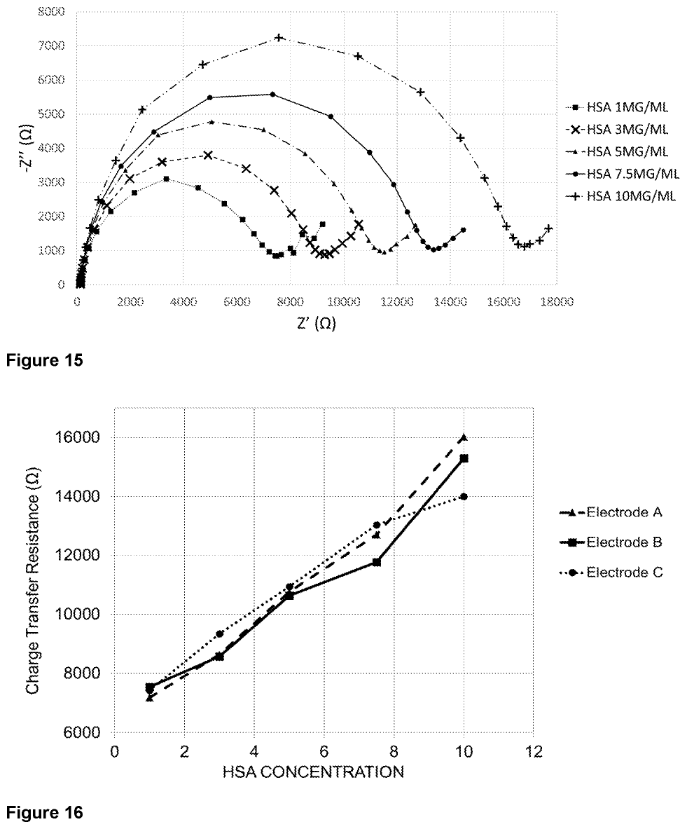

The test cassette is inserted into the device as seen in FIG. 15 that would apply the appropriate electrical parameters to record, calculate, and display the test results on the screen. The device's functionality sets and applies the desired volts, amplitude, frequency, and current needed to electrochemically measure the concentration.

In some embodiments, the device measures the ratio of GA to albumin and the ratio of albumin to creatinine. The data output displays GA/albumin ratio and albumin/creatinine ratio on the screen and the results are stored in memory of the reader.

In some embodiments, the device automatically save and transfer test results to a mobile device, app, or computer in which the recipient may be the user and/or a healthcare provider. The mode of transfer may be via a wireless or cable based connection.

In other embodiments, the device is also able to show the trend analysis of the results obtained in order to be able to track the progress of the patient's glucose control and/or kidney function.

Calibration:

There are separate calibration cassettes for each of the analytes. The calibration cassette is inserted into the instrument and different standard solutions of GA and albumin/creatinine are then added onto the calibration cassette. Using the calibrator standard solutions, a standard curve is generated and stored in the memory of the instrument reader. The calibration is performed before first use of the instrument and can be repeated as necessary, using standard calibration cassette and calibrator standard solutions. When a patient sample is added inside the cassette, the change in impedance is correlated with a stored calibration curve (obtained using standard solutions), and GA/albumin and albumin/creatinine ratios are determined. The GA/albumin ratio and albumin/creatinine ratio are then displayed on the screen by the reader.

Data Output:

The cassette is inserted into a handheld device as seen in FIG. 11 that has the appropriate electrical parameters to record, calculate, and finally display the GA/albumin ratio and albumin/creatinine ratio on the screen. The device's functionality sets and applies the desired volts, amplitude, frequency, and current needed to electrochemically measure the concentration. The device measures the GA, albumin, and creatinine concentration by measuring the change in impedance and correlating it with the calibration curve. The data output displays GA/albumin ratio and albumin/creatinine ratio on the screen and the results/data are stored in memory of the reader. The reported results can be downloaded and transferred to medical providers. It will be also able to show trend analysis of results obtained and can advise or comment based on results obtained.

### EXAMPLES

### Example 1. Electrochemical Detection of Various Human Serum Albumin Standards

Electrochemical detection of various human serum albumin (HSA) standards was performed using AUTOLAB (PGSTAT302N) instrument and analysis was performed using NOVA software. The experiment was conducted using ferric/ferrocyanide in phosphate buffer saline pH 7.4 as the redox probe. The three electrodes in the electrochemical cell comprise gold as the working electrode (A), platinum as the counter (auxiliary) electrode (B), and Ag/AgCl as the reference electrode (C). Impedance data was obtained by NOVA software and data was plotted as Nyquist plot which is a plot of Z(real) vs. Z(imaginary).

Working Electrode Treatment and Measurement:

The gold working electrode was treated with dithiobis(succinimidyl proprionate) (DSP, a covalent linker) which aids in immobilizing HSA antibodies on the gold working electrode. Further different concentrations of HSA were incubated and EIS was measured in ferric/ferrocyanide prepared in PBS. Molecules that attach to the gold electrode impede the electron transfer between the electrolyte solution and the gold electrode, leading to an increase in charge transfer resistance (Rct). The measurement of Rct was performed for various concentrations of HSA using NOVA software, and results were plotted in form of standard graph of charge transfer resistance vs. various concentrations of HSA.

FIG. 15 depicts the overlapping Nyquist curve for different concentrations of standard HSA i.e. 1 mg/ml, 3 mg/ml, 5 mg/ml, 7.5 mg/ml and 10 mg/ml. The Nyquist curve is a plot of Z real (on x-axis) vs. Z imaginary (on y-axis). It reveals an increasing semi-circle curve with increasing concentration of HSA. The results can be plotted as graph of charge transfer resistance i.e. delta Rct vs. HSA concentration.

FIG. 16 depicts the overlapping graph of charge transfer resistance Rct (in Ohms) and standard HSA concentration (mg/ml) for three electrodes A, B and C. The overlapping graph reveals that all the three electrodes depict good reproducibility for different HSA concentration i.e. 1 mg/ml, 3 mg/ml, 5 mg/ml, 7.5 mg/ml and 10 mg/ml. The graph reveals that the impedance measurement results for HSA were linear from concentration range of 1 mg/ml to 10 mg/ml.

### Example 2. Electrochemical Detection of Various Standards of Creatinine

Electrochemical detection of various creatinine standards was performed using AUTOLAB (PGSTAT302N) instrument and analysis was performed using NOVA software. The experiment was conducted using ferric/ferro cyanide in phosphate buffer saline pH 7.4 as the redox probe. The three electrodes in electrochemical cell comprise gold as the working electrode (A), platinum as the counter (auxiliary) electrode (B), and Ag/AgCl as the reference electrode (CA). Impedance data was obtained by NOVA software and data was plotted as Nyquist plot.

Working Electrode Treatment and Measurement:

Gold working electrode was treated with DSP, a covalent linker which immobilizes creatinine antibodies on the gold working electrode. Different concentrations of creatinine were incubated and EIS was measured. Molecules that attach to the gold electrode impede the electron transfer between the electrolyte solution and the gold electrode, leading to an increase in Rct. The measurement of Rct with Nyquist plot was performed for various concentration of creatinine using NOVA software, and the results were plotted in form of standard graph of charge transfer resistance vs. various concentrations of creatinine.

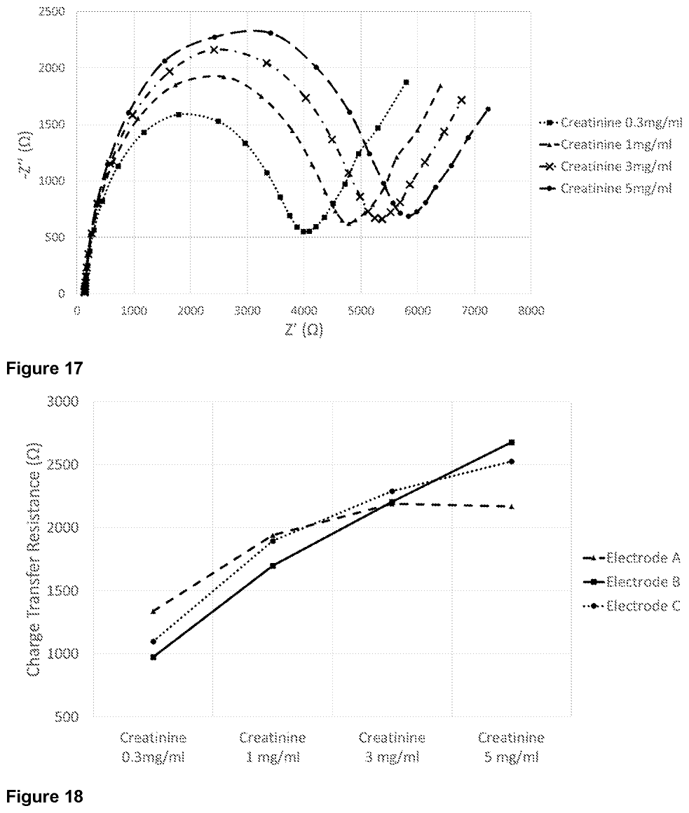

FIG. 17 depicts the overlapping Nyquist curves for different concentrations of creatinine standards i.e. 0.3 mg/ml, 1 mg/ml, 3 mg/ml and 5 mg/ml. The Nyquist curve is a plot of Z real (on x-axis) vs. Z imaginary (on y-axis). It reveals an increasing semi-circle curve with an increasing concentration of creatinine. The results can be plotted as a graph of charge transfer resistance i.e. delta Rct and creatinine concentration.

FIG. 18 depicts the overlapping graph of charge transfer resistance Rct (in Ohms), and creatinine standard concentration (mg/ml) for three electrodes A, B, and C. The overlapping graph reveals that all the three electrodes depict good reproducibility between electrodes for different creatinine concentration i.e., 1 mg/ml, 3 mg/ml, 5 mg/ml, 7.5 mg/ml and 10 mg/ml. The graph reveals that the impedance measurement results for creatinine were linear from the concentration range of 0.3 mg/ml to 5 mg/ml.

The graph of charge transfer resistance vs. different standard concentrations of HSA, GA or creatinine solutions may be stored in memory of the reader as standard graph. The amount of concentration of HSA, GA or creatinine in the sample can thus be deduced from the standard graph.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A device for measuring glucose control and kidney function, wherein the device measures both the ratio of glycated albumin to total albumin in a blood sample and the ratio of albumin to creatinine in a urine sample.

2. The device according to claim 1, comprising a microfluidic cassette which provides the reagents and sensing electrodes for testing the sample, and a measuring instrument that measures the change in electrical impedance caused by the test sample and calculates and reports a result.

3. The portable device according to claim 2, wherein the microfluidic cassette comprises two blood testing channels wherein one channel has embedded therein a glycated albumin sensing electrode and the other channel has embedded therein a total albumin sensing electrode; and wherein the measuring instrument measures the ratio of glycated albumin to total albumin in the blood sample.

4. The portable device according to claim 2, wherein the microfluidic cassette comprises two urine testing channels wherein one channel has embedded therein an albumin sensing electrode and the other channel has embedded a creatinine sensing electrode; and wherein the measuring instrument measures the ratio of albumin to creatinine in the urine sample.

5. The portable device according to claim 2, wherein the microfluidic cassette comprises four channels, wherein two channels test for glycated albumin and total albumin in a blood sample; and wherein the other two channels test for albumin and creatinine in a urine sample; and wherein the measuring instrument measures the ratio of glycated albumin to total albumin in the blood sample and also the ratio of albumin to creatinine in the urine sample.

6. The portable device according to claim 2, wherein the microfluidic cassette comprises one blood testing channel with two embedded electrodes; wherein one electrode is a glycated albumin sensing electrode and the other electrode is a total albumin sensing electrode; and wherein the measuring instrument measures the ratio of glycated albumin to total albumin in the blood sample.

7. The portable device according to claim 2, said microfluidic cassette comprises of one channel with two embedded electrodes; wherein one electrode is an albumin sensing electrode and the other electrode is a creatinine sensing electrode; and wherein the measuring instrument measures the ratio of albumin to creatinine in the urine sample.

8. The portable device according to claim 2, wherein the microfluidic cassette comprises two urine testing channels wherein each channel has two embedded electrodes and wherein in one channel the electrodes detect glycated albumin and total albumin respectively in a blood sample; whereas in the other channel the electrodes detect albumin and creatinine respectively in a urine sample.

9. The portable device according to claim 2, wherein each electrode is coated with a binding agent that will bind to a specific analyte in the test sample wherein the binding agent is an antibody or a binding peptide or an aptamer.

10. The portable device according to claim 7, wherein the sensing electrode is first coated with avidin and a biotinylated binding agent is then linked to the avidin.

11. The portable device according to claim 2, wherein the measuring instrument measures and calculates the results, provides reference values and trend analysis, and transmits the data to doctors and other healthcare providers.

12. A device for measuring glucose control and kidney function comprising:
a microfluidic cassette comprising an inlet including two channels, one or more reagents, one or more binding agents, and one or more electrodes;
a fluid sample; and
a reader wherein the microfluidic cassette is inserted into the reader and displays a ratio of glycated albumin to total albumin in the fluid sample by measuring changes in impedance.

13. The device according to claim 12, wherein the fluid sample is deposited into the inlet and configured to pass through the two channels.

14. The device according to claim 13, wherein the two channels include one or more reagents and one channel is configured to measure the glycated albumin and the other channel is configured to measure the albumin in the fluid sample.

15. The device according to claim 14, wherein the two channels propel the fluid sample forward until it reaches the electrodes which are at the distal end of each channel.

16. The device according to claim 15, wherein the electrodes include one or more binding agents.

17. The device according to claim 16, wherein the one or more binding agents are specific for glycated albumin and albumin.

18. The device according to claim 17, wherein a reaction between the fluid sample, one or more binding agents, and one or more reagents generate a change in impedance which is measured to display the ratio of glycated albumin to total albumin.
